(19) United States
(12) Patent Application Publication
(10) Pub. No.: US 2009/0069402 A1
(43) Pub. Date: Mar. 12, 2009

(54) DEUTERIUM-ENRICHED ATRASENTAN

(75) Inventor: Anthony W. Czarnik, Reno, NV (US)

Correspondence Address: VANCE INTELLECTUAL PROPERTY, PC, 5467 HILL TOP STREET, CROZET, VA, 22932-3167, US

(73) Assignee: PROTIA, LLC, Reno, NV (US)

(21) Appl. No.: 12/196,176

(22) Filed: Aug. 21, 2008

Related U.S. Application Data
(60) Provisional application No. 60/971,560, filed on Sep. 11, 2007.

(51) Int. Cl. A61K 31/4025; C07D 405/04; A61P 35/04

(52) U.S. Cl. 514/422; 548/526

(57) ABSTRACT

The present application describes deuterium-enriched atrasentan, pharmaceutically acceptable salt forms thereof, and methods of treating using the same.

### CROSS-REFERENCE TO RELATED APPLICATIONS

The present application claims priority benefit under 35 U.S.C. §119(e) of U.S. Provisional Patent Application Ser. No. 60/971,560 filed 11 Sep. 2007. The disclosure of this application is incorporated herein by reference.

### FIELD OF THE INVENTION

This invention relates generally to deuterium-enriched atrasentan, pharmaceutical compositions containing the same, and methods of using the same.

### BACKGROUND OF THE INVENTION

Atrasentan, shown below, is a well known selective endothelin-A receptor antagonist.

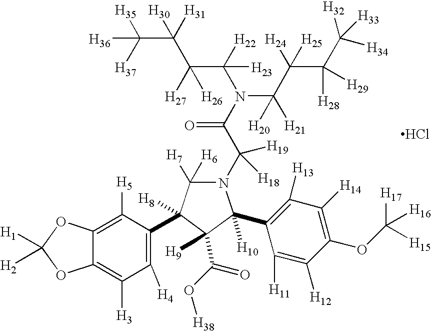

Since atrasentan is a known and useful pharmaceutical, it is desirable to discover novel derivatives thereof. Atrasentan is described in U.S. Pat. No. 5,767,144; the contents of which are incorporated herein by reference.

### SUMMARY OF THE INVENTION

Accordingly, one object of the present invention is to provide deuterium-enriched atrasentan or a pharmaceutically acceptable salt thereof.

It is another object of the present invention to provide pharmaceutical compositions comprising a pharmaceutically acceptable carrier and a therapeutically effective amount of at least one of the deuterium-enriched compounds of the present invention or a pharmaceutically acceptable salt thereof.

It is another object of the present invention to provide a method for treating prostate cancer, comprising administering to a host in need of such treatment a therapeutically effective amount of at least one of the deuterium-enriched compounds of the present invention or a pharmaceutically acceptable salt thereof.

It is another object of the present invention to provide a novel deuterium-enriched atrasentan or a pharmaceutically acceptable salt thereof for use in therapy.

It is another object of the present invention to provide the use of a novel deuterium-enriched atrasentan or a pharmaceutically acceptable salt thereof for the manufacture of a medicament (e.g., for the treatment of prostate cancer).

These and other objects, which will become apparent during the following detailed description, have been achieved by the inventor's discovery of the presently claimed deuterium-enriched atrasentan.

### DETAILED DESCRIPTION OF PREFERRED EMBODIMENTS

Deuterium (D or ²H) is a stable, non-radioactive isotope of hydrogen and has an atomic weight of 2.0144. Hydrogen naturally occurs as a mixture of the isotopes ¹H (hydrogen or protium), D (²H or deuterium), and T (³H or tritium). The natural abundance of deuterium is 0.015%. One of ordinary skill in the art recognizes that in all chemical compounds with a H atom, the H atom actually represents a mixture of H and D, with about 0.015% being D. Thus, compounds with a level of deuterium that has been enriched to be greater than its natural abundance of 0.015%, should be considered unnatural and, as a result, novel over their non-enriched counterparts.

All percentages given for the amount of deuterium present are mole percentages.

It can be quite difficult in the laboratory to achieve 100% deuteration at any one site of a lab scale amount of compound (e.g., milligram or greater). When 100% deuteration is recited or a deuterium atom is specifically shown in a structure, it is assumed that a small percentage of hydrogen may still be present. Deuterium-enriched can be achieved by either exchanging protons with deuterium or by synthesizing the molecule with enriched starting materials.

The present invention provides deuterium-enriched atrasentan or a pharmaceutically acceptable salt thereof. There are thirty-eight hydrogen atoms in the atrasentan portion of atrasentan as show by variables R₁-R₃₈ in formula I below.

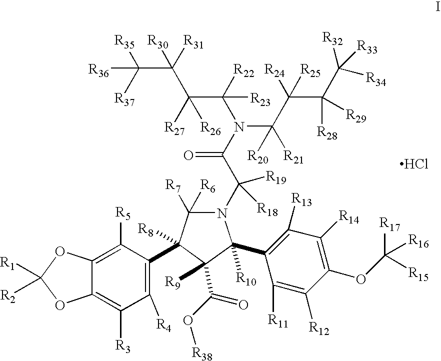

The hydrogens present on atrasentan have different capacities for exchange with deuterium. The hydrogen represented by R₃₈ is easily exchangeable with deuterium under physiological conditions and could be enriched by stirring with D₂O. Thus, when R₃₈ is a deuterium atom, it is expected that it will readily exchange with a proton after administration to a patient. The hydrogens represented by R₉, R₁₈, and R₁₉ are exchangeable with deuterium in the presence of a strong acid (e.g., D₂SO₄/D₂O) or strong base (e.g., LiO-t-Bu/DO-t-Bu). It should be possible to arrive experimentally at acidic or basic conditions that allow the exchange of R₉, R₁₈, and R₁₉ without decomposition of the atrasentan molecule. It is recognized that the stereocenter bearing R₉ may undergo epimerization under these conditions, but it is likely that, based on steric grounds, useful amounts of the desired stereoisomer will be produced. Treatment with strong acid (e.g., D₂SO₄/D₂O) may cause the exchange of R₃-R₅ and R₁₁-R₁₄ for deuterium. Since atrasentan contains potentially acid sensitive acetal and amide groups, exchange of R₃-R₅ and R₁₁-R₁₄ with deuterium may occur with varying amounts of hydrolysis of the acetal and amide groups. Nonetheless, it may be possible to arrive experimentally at suitable conditions to cause an acid-catalyzed exchange of R₃-R₅ and R₁₁-R₁₄ for deuterium without causing significant amounts of decomposition of the atrasentan molecule. The hydrogens represented by R₁-R₂, R₆-R₈, R₁₀, R₁₅-R₁₇, and R₂₀, R₃₇ are non-exchangeable. Deuterium enrichment can only reasonably occur by using deuterated reagents during the synthesis of atrasentan hydrochloride or by partial degradation of atrasentan followed by reinstalling various groups.

The present invention is based on increasing the amount of deuterium present in atrasentan above its natural abundance. This increasing is called enrichment or deuterium-enrichment. If not specifically noted, the percentage of enrichment refers to the percentage of deuterium present in the compound, mixture of compounds, or composition. Examples of the amount of enrichment include from about 0.5, 1, 2, 3, 4, 5, 6, 7, 8, 9, 10, 12, 16, 21, 25, 29, 33, 37, 42, 46, 50, 54, 58, 63, 67, 71, 75, 79, 84, 88, 92, 96, to about 100 mol %. Since there are 38 hydrogens in atrasentan, replacement of a single hydrogen atom with deuterium would result in a molecule with about 3% deuterium enrichment. In order to achieve enrichment less than about 3%, but above the natural abundance, only partial deuteration of one site is required. Thus, less than about 3% enrichment would still refer to deuterium-enriched atrasentan.

With the natural abundance of deuterium being 0.015%, one would expect that for approximately every 6,667 molecules of atrasentan (1/0.00015=6,667), there is one naturally occurring molecule with one deuterium present. Since atrasentan has 38 positions, one would roughly expect that for approximately every 253,346 molecules of atrasentan (38×6,667), all 38 different, naturally occurring, mono-deuterated atrasentans would be present. This approximation is a rough estimate as it doesn't take into account the different exchange rates of the hydrogen atoms on atrasentan. For naturally occurring molecules with more than one deuterium, the numbers become vastly larger. In view of this natural abundance, the present invention, in an embodiment, relates to an amount of an deuterium enriched compound, whereby the enrichment recited will be more than naturally occurring deuterated molecules.

In view of the natural abundance of deuterium-enriched atrasentan, the present invention also relates to isolated or purified deuterium-enriched atrasentan. The isolated or purified deuterium-enriched atrasentan is a group of molecules whose deuterium levels are above the naturally occurring levels (e.g., 3%). The isolated or purified deuterium-enriched atrasentan can be obtained by techniques known to those of skill in the art (e.g., see the syntheses described below).

The present invention also relates to compositions comprising deuterium-enriched atrasentan. The compositions require the presence of deuterium-enriched atrasentan which is greater than its natural abundance. For example, the compositions of the present invention can comprise (a) a μg of a deuterium-enriched atrasentan; (b) a mg of a deuterium-enriched atrasentan; and, (c) a gram of a deuterium-enriched atrasentan.

In an embodiment, the present invention provides an amount of a novel deuterium-enriched atrasentan.

Examples of amounts include, but are not limited to (a) at least 0.01, 0.02, 0.03, 0.04, 0.05, 0.1, 0.2, 0.3, 0.4, 0.5, to 1 mole, (b) at least 0.1 moles, and (c) at least 1 mole of the compound. The present amounts also cover lab-scale (e.g., gram scale), kilo-lab scale (e.g., kilogram scale), and industrial or commercial scale (e.g., multi-kilogram or above scale) quantities as these will be more useful in the actual manufacture of a pharmaceutical. Industrial/commercial scale refers to the amount of product that would be produced in a batch that was designed for clinical testing, formulation, sale/distribution to the public, etc.

In another embodiment, the present invention provides a novel, deuterium enriched compound of formula I or a pharmaceutically acceptable salt thereof.

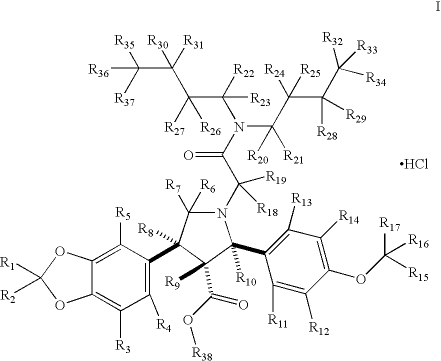

wherein R₁-R₃₈ are independently selected from H and D; and the abundance of deuterium in R₁-R₃₈ is at least 3%. The abundance can also be (a) at least 5%, (b) at least 11%, (c) at least 16%, (d) at least 21%, (e) at least 26%, (f) at least 32%, (g) at least 37%, (h) at least 42%, (i) at least 47%, (j) at least 53%, (k) at least 58%, (l) at least 63%, (m) at least 68%, (n) at least 74%, (o) at least 79%, (p) at least 84%, (q) at least 89%, (r) at least 95%, and (s) 100%.

In another embodiment, the present invention provides a novel, deuterium enriched compound of formula I or a pharmaceutically acceptable salt thereof, wherein the abundance of deuterium in R₉, R₁₈, and R₁₉ is at least 33%. The abundance can also be (a) at least 67%, and (b) 100%.

In another embodiment, the present invention provides a novel, deuterium enriched compound of formula I or a pharmaceutically acceptable salt thereof, wherein the abundance of deuterium in R₃-R₅, and R₁₁-R₁₄ is at least 14%. The abundance can also be (a) at least 29%, (b) at least 43%, (c) at least 57%, (d) at least 71%, (e) at least 3086%, and (f) 100%.

In another embodiment, the present invention provides a novel, deuterium enriched compound of formula I or a pharmaceutically acceptable salt thereof, wherein the abundance of deuterium in R₁-R₂, R₆-R₈, R₁₀, R₁₅-R₁₇, R₂₀, and R₃₇ is at least 9%. The abundance can also be (a) at least 18%, (b) at least 27%, (c) at least 36%, (d) at least 45%, (e) at least 56%, (f) at least 64%, (g) at least 73%, (h) at least 82%, (i) at least 91%, and (j) 100%.

In another embodiment, the present invention provides a novel, deuterium enriched compound of formula I, wherein the abundance of deuterium in R₃-R₅, R₉, R₁₁-R₁₄, R₁₈, R₁₉ is at least 10%. The abundance can also be (a) at least 20%, (b) at least 30%, (c) at least 40%, (d) at least 50%, (e) at least 60%, (f) at least 70%, (g) at least 80%, (h) at least 90%, and (i) 100%.

In another embodiment, the present invention provides a novel, deuterium enriched compound of formula I or a pharmaceutically acceptable salt thereof, wherein the abundance of deuterium in R₁-R₂, R₆-R₈, R₉, R₁₀, R₁₅-R₁₇, R₁₈, R₁₉, R₂₀, and R₃₈ is at least 7%. The abundance can also be (a) at least 14%, (b) at least 21%, (c) at least 29%, (d) at least 36%, (e) at least 43%, (f) at least 50%, (g) at least 57%, (h) at least 64%, (i) at least 71%, (j) at least 79%, (k) at least 86%, (l) at least 93%, and (m) 100%.

In another embodiment, the present invention provides a novel, deuterium enriched compound of formula I or a pharmaceutically acceptable salt thereof, wherein the abundance of deuterium in R₁-R₂, R₃-R₅, R₆-R₈, R₁₀, R₁₁-R₁₄, R₁₅-R₁₇, R₂₀, and R₃₇ is at least 6%. The abundance can also be (a) at least 11%, (b) at least 17%, (c) at least 22%, (d) at least 28%, (e) at least 33%, (f) at least 39%, (g) at least 44%, (h) at least 50%, (i) at least 56%, (j) at least 61%, (k) at least 67%, (l) at least 72%, (m) at least 78%, (n) at least 83%, (o) at least 89%, (p) at least 94%, and (q) 100%.

In another embodiment, the present invention provides a novel, deuterium enriched compound of formula I or a pharmaceutically acceptable salt thereof, wherein the abundance of deuterium in R₁-R₂ is at least 50%. The abundance can also be (a) at least 100%.

In another embodiment, the present invention provides a novel, deuterium enriched compound of formula I or a pharmaceutically acceptable salt thereof, wherein the abundance of deuterium in R₁₅-R₁₇ is at least 33%. The abundance can also be (a) at least 67%, and (b) 100%.

In another embodiment, the present invention provides a novel, deuterium enriched compound of formula I or a pharmaceutically acceptable salt thereof, wherein the abundance of deuterium in R₂₀-R₃₇ is at least 6%. The abundance can also be (a) at least 11%, (b) at least 17%, (c) at least 22%, (d) at least 28%, (e) at least 33%, (f) at least 39%, (g) at least 44%, (h) at least 50%, (i) at least 56%, (j) at least 61%, (k) at least 67%, (l) at least 72%, (m) at least 78%, (n) at least 83%, (o) at least 89%, (p) at least 94%, and (q) 100%.

In another embodiment, the present invention provides an isolated novel, deuterium enriched compound of formula I or a pharmaceutically acceptable salt thereof.

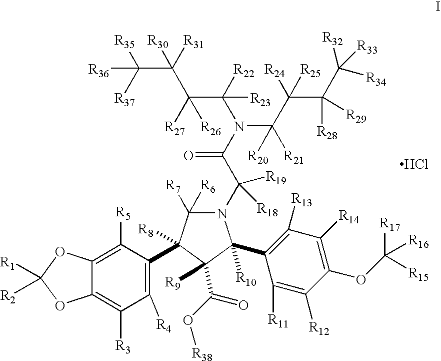

wherein R₁-R₃₈ are independently selected from H and D; and the abundance of deuterium in R₁-R₃₈ is at least 3%. The abundance can also be (a) at least 5%, (b) at least 11%, (c) at least 16%, (d) at least 21%, (e) at least 26%, (f) at least 32%, (g) at least 37%, (h) at least 42%, (i) at least 47%, (j) at least 53%, (k) at least 58%, (l) at least 63%, (m) at least 68%, (n) at least 74%, (o) at least 79%, (p) at least 84%, (q) at least 89%, (r) at least 95%, and (s) 100%.

In another embodiment, the present invention provides an isolated novel, deuterium enriched compound of formula I or a pharmaceutically acceptable salt thereof, wherein the abundance of deuterium in R₉, R₁₈, and R₁₉ is at least 33%. The abundance can also be (a) at least 67%, and (b) 100%.

In another embodiment, the present invention provides an isolated novel, deuterium enriched compound of formula I or a pharmaceutically acceptable salt thereof, wherein the abundance of deuterium in R₃-R₅, and R₁₁-R₁₄ is at least 14%. The abundance can also be (a) at least 29%, (b) at least 43%, (c) at least 57%, (d) at least 71%, (e) at least 3086%, and (f) 100%.

In another embodiment, the present invention provides an isolated novel, deuterium enriched compound of formula I or a pharmaceutically acceptable salt thereof, wherein the abundance of deuterium in R₁-R₂, R₆-R₈, R₁₀, R₁₅-R₁₇, R₂₀, and R₃₇ is at least 9%. The abundance can also be (a) at least 18%, (b) at least 27%, (c) at least 36%, (d) at least 45%, (e) at least 56%, (f) at least 64%, (g) at least 73%, (h) at least 82%, (i) at least 91%, and (j) 100%.

In another embodiment, the present invention provides an isolated novel, deuterium enriched compound of formula I, wherein the abundance of deuterium in R₃-R₅, R₉, R₁₁-R₁₄, R₁₈, R₁₉ is at least 10%. The abundance can also be (a) at least 20%, (b) at least 30%, (c) at least 40%, (d) at least 50%, (e) at least 60%, (f) at least 70%, (g) at least 80%, (h) at least 90%, and (i) 100%.

In another embodiment, the present invention provides an isolated novel, deuterium enriched compound of formula I or a pharmaceutically acceptable salt thereof, wherein the abundance of deuterium in R₁-R₂, R₆-R₈, R₉, R₁₀, R₁₅-R₁₇, R₁₈, R₁₉, R₂₀, and R₃₇ is at least 7%. The abundance can also be (a) at least 14%, (b) at least 21%, (c) at least 29%, (d) at least 36%, (e) at least 43%, (f) at least 50%, (g) at least 57%, (h) at least 64%, (i) at least 71%, (j) at least 79%, (k) at least 86%, (l) at least 93%, and (m) 100%.

In another embodiment, the present invention provides an isolated novel, deuterium enriched compound of formula I or a pharmaceutically acceptable salt thereof, wherein the abundance of deuterium in R₁-R₂, R₃-R₅, R₆-R₈, R₁₀, R₁₁-R₁₄, R₁₅-R₁₇, R₂₀, and R₃₇ is at least 6%. The abundance can also be (a) at least 11%, (b) at least 17%, (c) at least 22%, (d) at least 28%, (e) at least 33%, (f) at least 39%, (g) at least 44%, (h) at least 50%, (i) at least 56%, (j) at least 61%, (k) at least 67%, (l) at least 72%, (m) at least 78%, (n) at least 83%, (o) at least 89%, (p) at least 94%, and (q) 100%.

In another embodiment, the present invention provides an isolated novel, deuterium enriched compound of formula I or a pharmaceutically acceptable salt thereof, wherein the abundance of deuterium in R₁-R₂ is at least 50%. The abundance can also be (a) at least 100%.

In another embodiment, the present invention provides an isolated novel, deuterium enriched compound of formula I or a pharmaceutically acceptable salt thereof, wherein the abundance of deuterium in R₁₅-R₁₇ is at least 33%. The abundance can also be (a) at least 67%, and (b) 100%.

In another embodiment, the present invention provides an isolated novel, deuterium enriched compound of formula I or a pharmaceutically acceptable salt thereof, wherein the abundance of deuterium in R₂₀-R₃₇ is at least 6%. The abundance can also be (a) at least 11%, (b) at least 17%, (c) at least 22%, (d) at least 28%, (e) at least 33%, (f) at least 39%, (g) at least 44%, (h) at least 50%, (i) at least 56%, (j) at least 61%, (k) at least 67%, (l) at least 72%, (m) at least 78%, (n) at least 83%, (o) at least 89%, (p) at least 94%, and (q) 100%.

In another embodiment, the present invention provides novel mixture of deuterium enriched compounds of formula I or a pharmaceutically acceptable salt thereof.

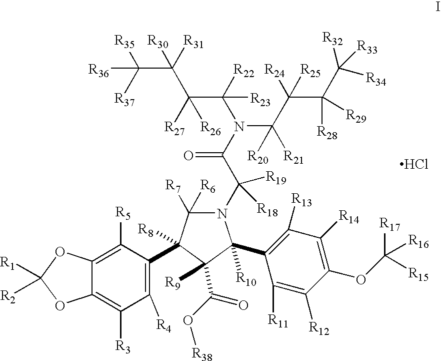

wherein R₁-R₃₈ are independently selected from H and D; and the abundance of deuterium in R₁-R₃₈ is at least 3%. The abundance can also be (a) at least 5%, (b) at least 11%, (c) at least 16%, (d) at least 21%, (e) at least 26%, (f) at least 32%, (g) at least 37%, (h) at least 42%, (i) at least 47%, (j) at least 53%, (k) at least 58%, (1) at least 63%, (m) at least 68%, (n) at least 74%, (o) at least 79%, (p) at least 84%, (q) at least 89%, (r) at least 95%, and (s) 100%.

In another embodiment, the present invention provides a novel mixture of, deuterium enriched compound of formula I or a pharmaceutically acceptable salt thereof, wherein the abundance of deuterium in R₉, R₁₈, and R₁₉ is at least 33%. The abundance can also be (a) at least 67%, and (b) 100%.

In another embodiment, the present invention provides a novel mixture of, deuterium enriched compound of formula I or a pharmaceutically acceptable salt thereof, wherein the abundance of deuterium in R₃-R₅, and R₁₁-R₁₄ is at least 14%. The abundance can also be (a) at least 29%, (b) at least 43%, (c) at least 57%, (d) at least 71%, (e) at least 3086%, and (f) 100%.

In another embodiment, the present invention provides a novel mixture of, deuterium enriched compound of formula I or a pharmaceutically acceptable salt thereof, wherein the abundance of deuterium in R₁-R₂, R₆-R₈, R₁₀, R₁₅-R₁₇, R₂₀, and R₃₇ is at least 9%. The abundance can also be (a) at least 18%, (b) at least 27%, (c) at least 36%, (d) at least 45%, (e) at least 56%, (f) at least 64%, (g) at least 73%, (h) at least 82%, (i) at least 91%, and (j) 100%.

In another embodiment, the present invention provides a novel mixture of, deuterium enriched compound of formula I, wherein the abundance of deuterium in R₃-R₅, R₉, R₁₁-R₁₄, R₁₈, R₁₉ is at least 10%. The abundance can also be (a) at least 20%, (b) at least 30%, (c) at least 40%, (d) at least 50%, (e) at least 60%, (f) at least 70%, (g) at least 80%, (h) at least 90%, and (i) 100%.

In another embodiment, the present invention provides a novel mixture of, deuterium enriched compound of formula I or a pharmaceutically acceptable salt thereof, wherein the abundance of deuterium in R₁-R₂, R₆-R₈, R₉, R₁₀, R₁₅-R₁₇, R₁₈, R₁₉, R₂₀, and R₃₇ is at least 7%. The abundance can also be (a) at least 14%, (b) at least 21%, (c) at least 29%, (d) at least 36%, (e) at least 43%, (f) at least 50%, (g) at least 57%, (h) at least 64%, (i) at least 71%, (j) at least 79%, (k) at least 86%, (l) at least 93%, and (m) 100%.

In another embodiment, the present invention provides a novel mixture of, deuterium enriched compound of formula I or a pharmaceutically acceptable salt thereof, wherein the abundance of deuterium in R₁-R₂, R₃-R₅, R₆-R₈, R₁₀, R₁₁-R₁₄, R₁₅-R₁₇, R₂₀, and R₃₇ is at least 6%. The abundance can also be (a) at least 11%, (b) at least 17%, (c) at least 22%, (d) at least 28%, (e) at least 33%, (f) at least 39%, (g) at least 44%, (h) at least 50%, (i) at least 56%, (j) at least 61%, (k) at least 67%, (l) at least 72%, (m) at least 78%, (n) at least 83%, (o) at least 89%, (p) at least 94%, and (q) 100%.

In another embodiment, the present invention provides a novel mixture of, deuterium enriched compound of formula I or a pharmaceutically acceptable salt thereof, wherein the abundance of deuterium in R₁-R₂ is at least 50%. The abundance can also be (a) at least 100%.

In another embodiment, the present invention provides a novel mixture of, deuterium enriched compound of formula I or a pharmaceutically acceptable salt thereof, wherein the abundance of deuterium in R₁₅-R₁₇ is at least 33%. The abundance can also be (a) at least 67%, and (b) 100%.

In another embodiment, the present invention provides a novel mixture of, deuterium enriched compound of formula I or a pharmaceutically acceptable salt thereof, wherein the abundance of deuterium in R₂₀-R₃₇ is at least 6%. The abundance can also be (a) at least 11%, (b) at least 17%, (c) at least 22%, (d) at least 28%, (e) at least 33%, (f) at least 39%, (g) at least 44%, (h) at least 50%, (i) at least 56%, (j) at least 61%, (k) at least 67%, (1) at least 72%, (m) at least 78%, (n) at least 83%, (o) at least 89%, (p) at least 94%, and (q) 100%.

In another embodiment, the present invention provides novel pharmaceutical compositions, comprising: a pharmaceutically acceptable carrier and a therapeutically effective amount of a deuterium-enriched compound of the present invention.

In another embodiment, the present invention provides a novel method for treating prostate cancer comprising: administering to a patient in need thereof a therapeutically effective amount of a deuterium-enriched compound of the present invention.

In another embodiment, the present invention provides an amount of a deuterium-enriched compound of the present invention as described above for use in therapy.

In another embodiment, the present invention provides the use of an amount of a deuterium-enriched compound of the present invention for the manufacture of a medicament (e.g., for the treatment of prostate cancer).

The present invention may be embodied in other specific forms without departing from the spirit or essential attributes thereof. This invention encompasses all combinations of preferred aspects of the invention noted herein. It is understood that any and all embodiments of the present invention may be taken in conjunction with any other embodiment or embodiments to describe additional more preferred embodiments. It is also to be understood that each individual element of the preferred embodiments is intended to be taken individually as its own independent preferred embodiment. Furthermore, any element of an embodiment is meant to be combined with any and all other elements from any embodiment to describe an additional embodiment.

### Definitions

The examples provided in the definitions present in this application are non-inclusive unless otherwise stated. They include but are not limited to the recited examples.

The compounds of the present invention may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. All processes used to prepare compounds of the present invention and intermediates made therein are considered to be part of the present invention. All tautomers of shown or described compounds are also considered to be part of the present invention.

“Host” preferably refers to a human. It also includes other mammals including the equine, porcine, bovine, feline, and canine families.

“Treating” or “treatment” covers the treatment of a disease-state in a mammal, and includes: (a) preventing the disease-state from occurring in a mammal, in particular, when such mammal is predisposed to the disease-state but has not yet been diagnosed as having it; (b) inhibiting the disease-state, e.g., arresting it development; and/or (c) relieving the disease-state, e.g., causing regression of the disease state until a desired endpoint is reached. Treating also includes the amelioration of a symptom of a disease (e.g., lessen the pain or discomfort), wherein such amelioration may or may not be directly affecting the disease (e.g., cause, transmission, expression, etc.).

“Therapeutically effective amount” includes an amount of a compound of the present invention that is effective when administered alone or in combination to treat the desired condition or disorder. “Therapeutically effective amount” includes an amount of the combination of compounds claimed that is effective to treat the desired condition or disorder. The combination of compounds is preferably a synergistic combination. Synergy, as described, for example, by Chou and Talalay, Adv. Enzyme Regul. 1984, 22:27-55, occurs when the effect of the compounds when administered in combination is greater than the additive effect of the compounds when administered alone as a single agent. In general, a synergistic effect is most clearly demonstrated at sub-optimal concentrations of the compounds. Synergy can be in terms of lower cytotoxicity, increased antiviral effect, or some other beneficial effect of the combination compared with the individual components.

“Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of the basic residues. The pharmaceutically acceptable salts include the conventional quaternary ammonium salts of the parent compound formed, for example, from non-toxic inorganic or organic acids. For example, such conventional non-toxic salts include, but are not limited to, those derived from inorganic and organic acids selected from 1,2-ethanedisulfonic, 2-acetoxybenzoic, 2-hydroxyethanesulfonic, acetic, ascorbic, benzenesulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methanesulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, and toluenesulfonic.

### Synthesis

Atrasentan hydrochloride can be prepared according to well-known procedures (see for example U.S. Pat. No. 5,767,144, J. Med. Chem. 1996, 39, 1039 and Tetrahedron Lett. 1999, 40, 7175). Scheme 1 shows an example of how to prepare atrasentan hydrochloride.

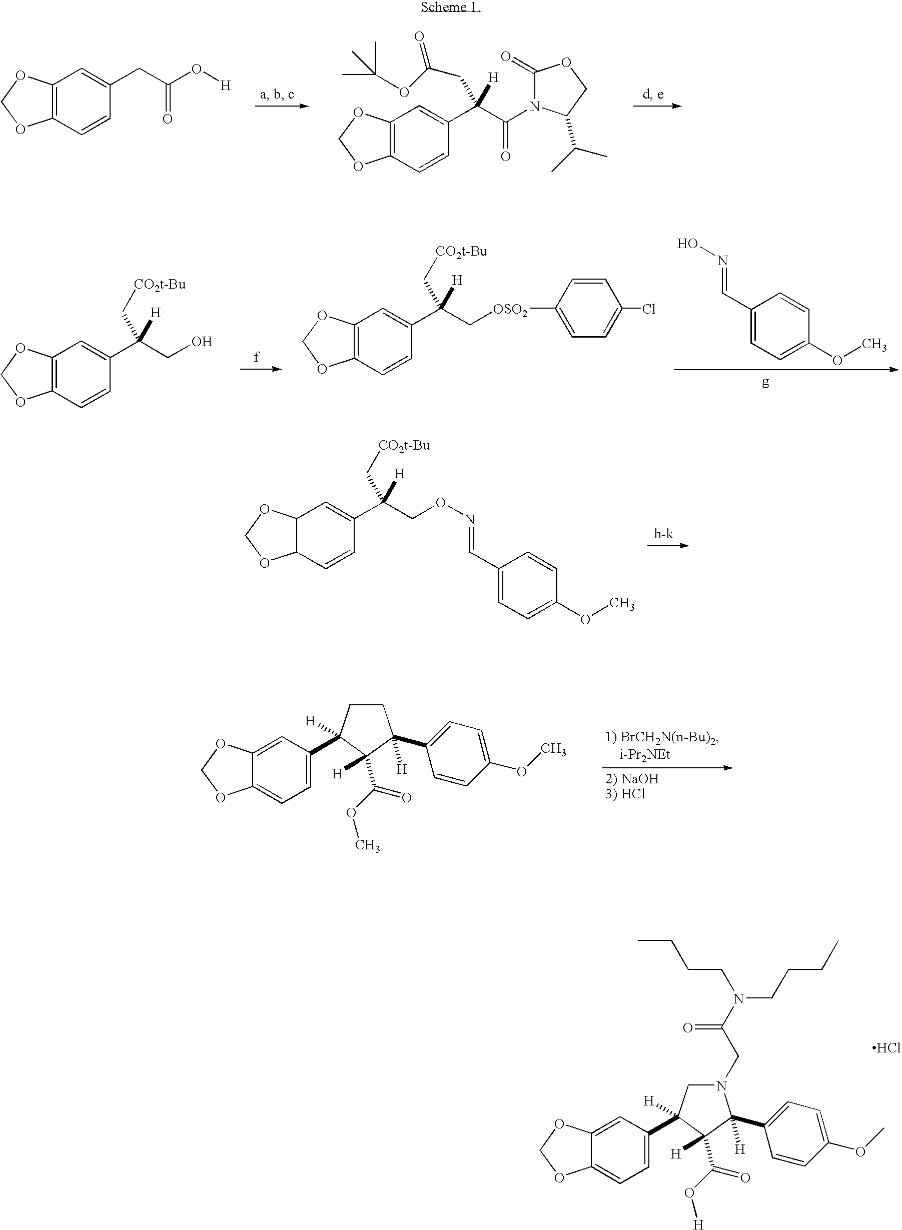

Atrasentan itself may be used as a starting material for the synthesis of deuterated versions (other than deuteration of the physiologically exchangeable hydrogen R₃₈ or by exchange of protium for deuterium using catalytic acids and bases, vide supra). Synthesis of atrasentan hydrochloride with deuterium in the dibutylamino group. Hydrolysis of atrasentan under acidic or basic aqueous conditions should produce a dicarboxylic acid that can be converted back to atrasentan hydrochloride as shown in Scheme 2 using fully or partially deuterated dibutylamine. It is recognized that there are two carboxylic acids that could couple with the deuterated dibutylamine, but the desired carboxylic acid should couple more quickly on steric grounds to provide deuterated atrasentan selectively. In this way, compounds such as atrasentan hydrochloride with R₂₀-R₃₇=deuterium may be made, as well as many analogs bearing fewer than the full complement of deuteria in the dibutylamino group, e.g., a compound with R₂₀-R₂₃=deuterium and R₂₄-R₃₇=hydrogen. The requisite diamine for the latter analog can be made by the reduction of (C₃H₇CO)₂NH with LiAlD₄. Other analogs bearing fewer than the full complement of deuteria in the dibutylamino group may be made from partially deuterated dibutylamines that may be made by a variety of standard routes.

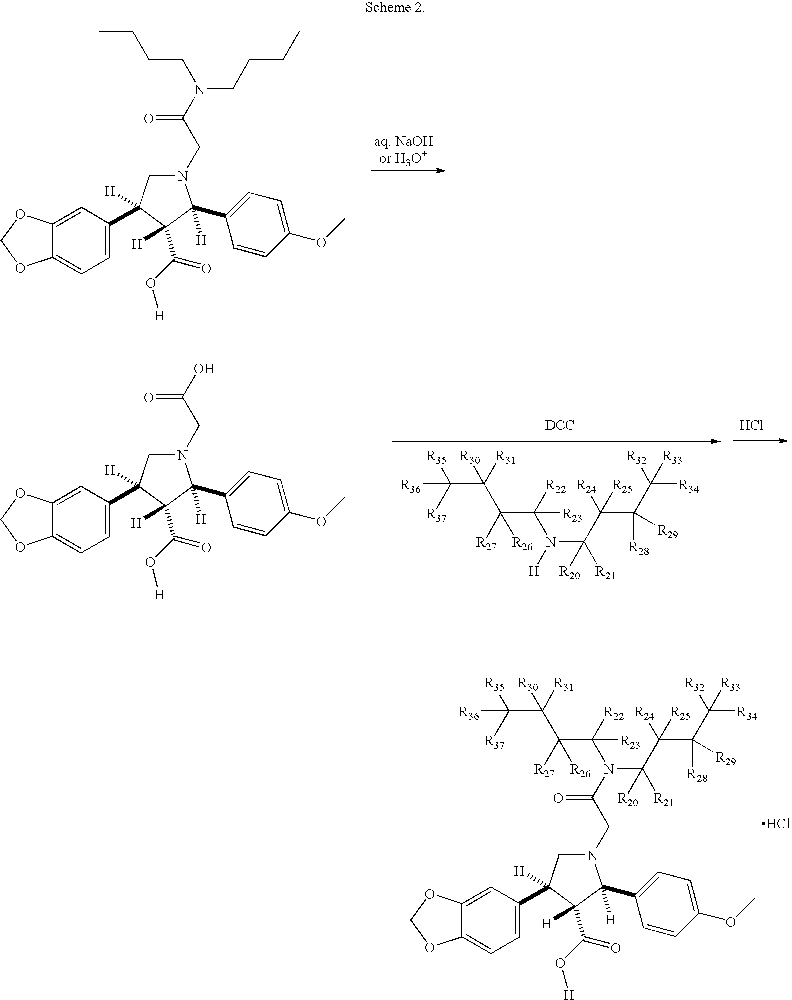

Synthesis of atrasentan hydrochloride with deuterium in the methyl group. Nucleophilic dealkylation of atrasentan (e.g., with BCl₃ or BBr₃, with NaCl in hot DMSO, or with LiSCH₃ in a dipolar, aprotic solvent such as HMPA or DMPU) should provide a phenol that can be alkylated with CD₃I or (CD₃)₂SO₄ (Scheme 3) to introduce a trideuteriomethyl ether. The carboxylic acid may also be alkylated to produce a trideuteriomethyl ester, but saponification at room temperature (to avoid hydrolysis of the amide) should hydrolyze the ester and produce atrasentan hydrochloride with R₁₅-R₁₇=deuterium after neutralization and hydrochloride formation.

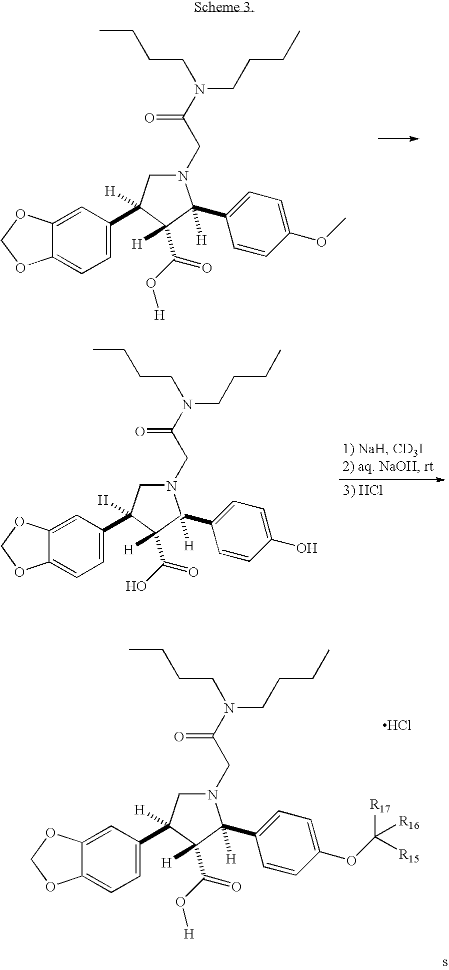

Synthesis of dideuterioalendronates. Examples of dideuterated 4-aminobutyric acids for making compounds of the present invention and suggested routes for making these materials are shown in Scheme 3. The product of the first route would be useful for making alendronate sodium where R⁶ and R⁷ are deuterium. The product of the second route would be useful for making alendronate sodium where R¹⁰ and R¹¹ are deuterium. The product of the second route would be useful for making alendronate sodium where R⁸ and R⁹ are deuterium. Schemes 4-6 show synthesis of deuterated atrasentan hydrochloride from atrasentan. The synthesis of atrasentan hydrochloride shown in Scheme 1 above offers several opportunities for incorporating deuterium during its preparation. In the figures below, a route to perdeuterated atrasentan is outlined. A person skilled in the art of organic synthesis would recognize that many partially deuterated analogs may also be prepared by replacing deuterium-bearing reagents or starting materials with protium-bearing counterparts. Scheme 4 shows a route to the perdeuterated carboxylic acid used in Scheme 1 above. To illustrate the idea that partially deuterated materials are also encompassed in the design, commercial piperonal (the second compound in the Figure except without deuterium) could be employed to arrive at a carboxylic acid bearing two fewer deuterium atoms. Alternatively, the dideuteriopiperonal shown could be used, but deuteration in the following steps could be avoided.

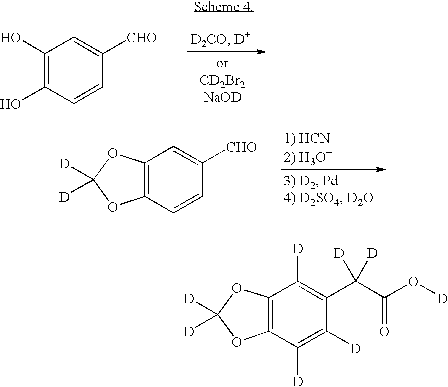

With the carboxylic acid from Scheme 4 in hand, further elaboration to the alcohol shown in Scheme 5 may be accomplished using the techniques of Scheme 1 except that deuterated reagents may be employed. Again, a combination of deuterated and non-deuterated reagents would produce a material with less than the full complement of deuterium atoms if desired.

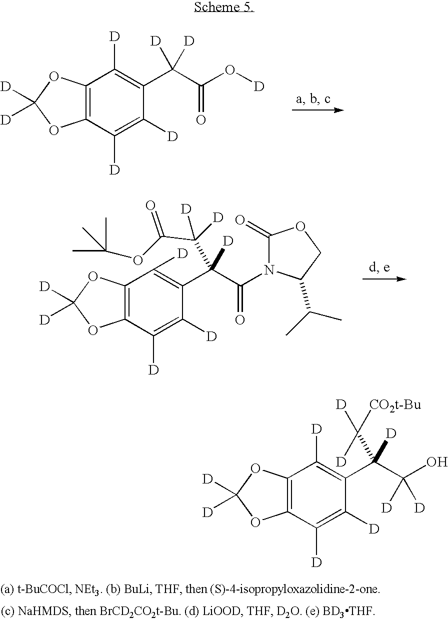

Scheme 6 shows the completion of the synthesis of perdeuterated atrasentan hydrochloride (physiologically labile deuterons have been replaced by protons). Again the methods of Scheme 1 are employed, except with deuterated reagents and starting materials. Partial deuteration may be achieved using reagents or starting materials that bear some combination of protons.

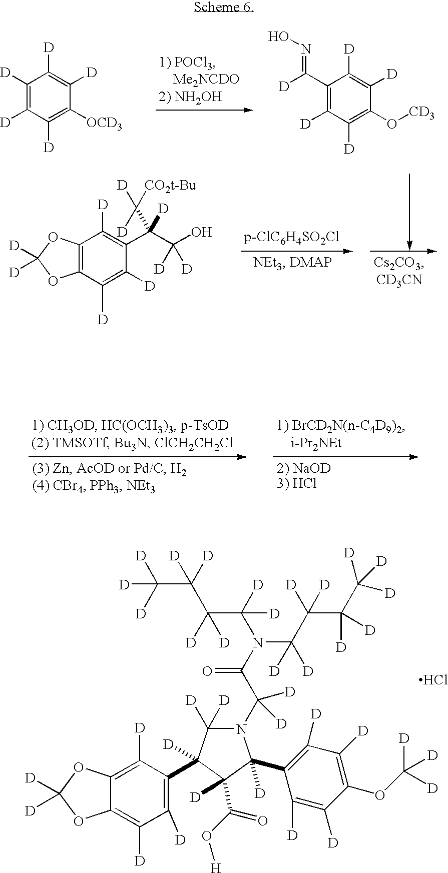

### EXAMPLES

Table 1 provides compounds that are representative examples of the present invention. When one of R₁-R₂₅ is present, it is selected from H or D.

| | |
|---|---|
| 1 | 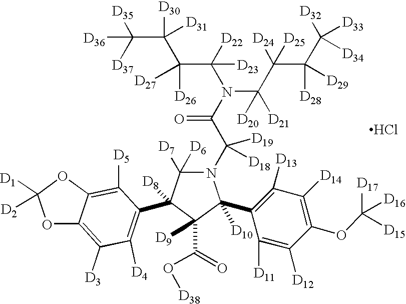 |
| 2 | 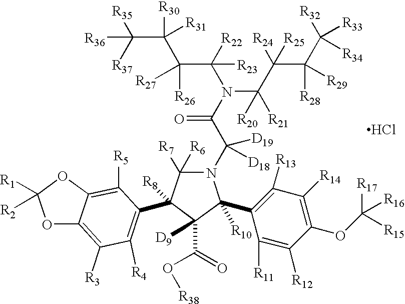 |
| 3 | 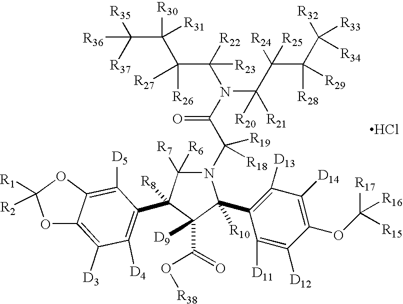 |
| 4 | 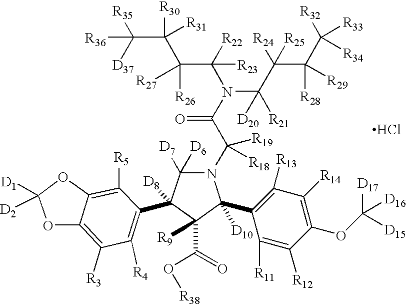 |
| 5 | 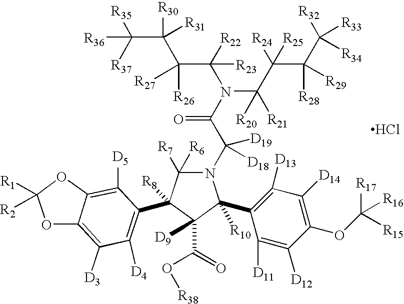 |
| 6 | 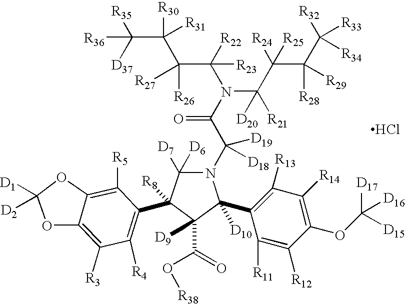 |
| 7 | 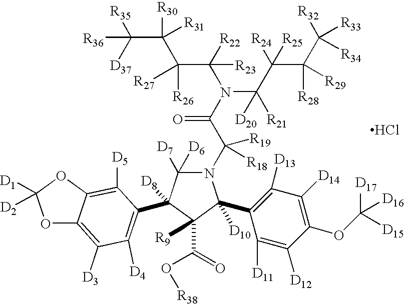 |
| 8 | 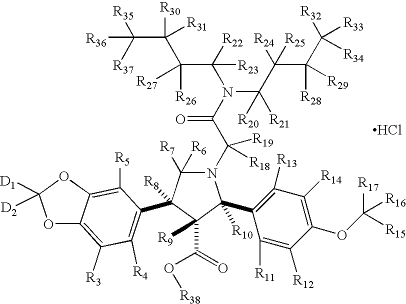 |
| 9 | 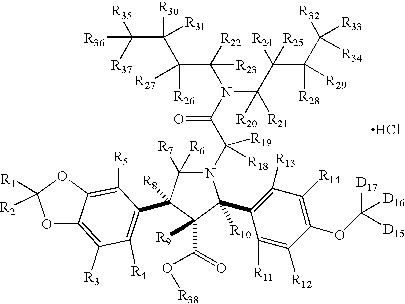 |
| 10 | 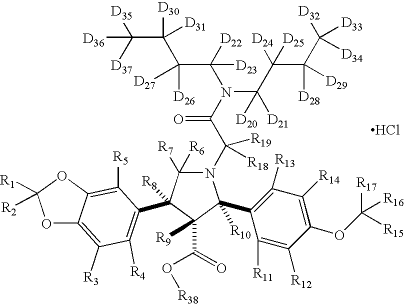 |

Table 2 provides compounds that are representative examples of the present invention. Where H is shown, it represents naturally abundant hydrogen.

| | |
|---|---|
| 11 | 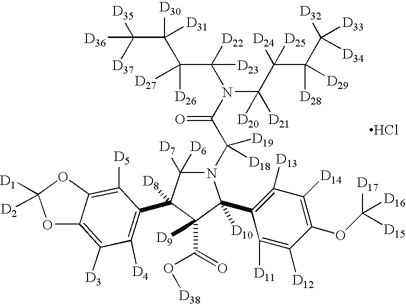 |
| 12 | 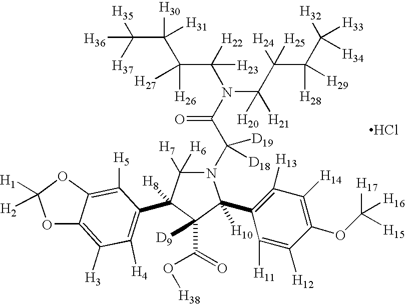 |
| 13 | 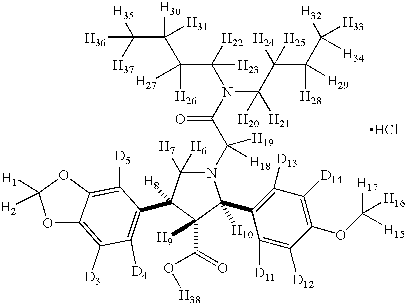 |
| 14 | 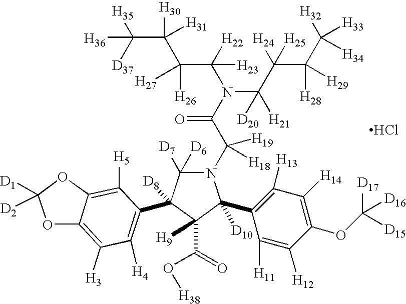 |
| 15 | 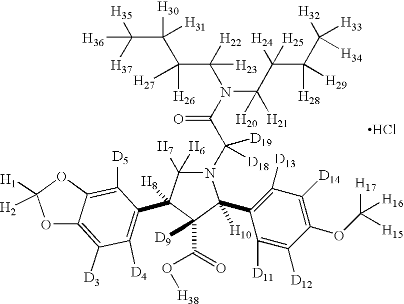 |
| 16 | 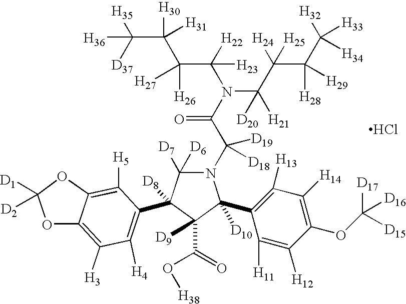 |
| 17 | 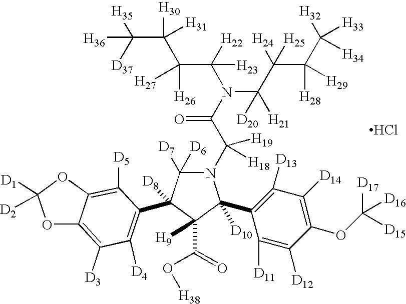 |
| 18 | 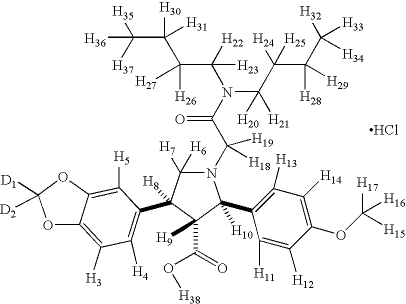 |
| 19 | 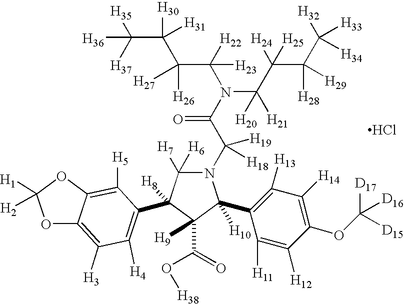 |
| 20 | 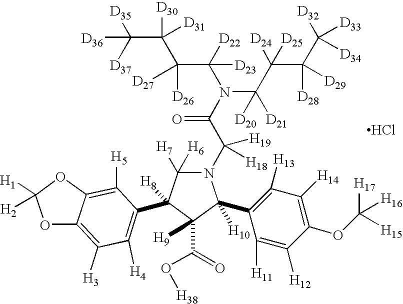 |

Numerous modifications and variations of the present invention are possible in light of the above teachings. It is therefore to be understood that within the scope of the appended claims, the invention may be practiced otherwise that as specifically described herein.

What is claimed is:

1. A deuterium-enriched compound of formula I or a pharmaceutically acceptable salt thereof: 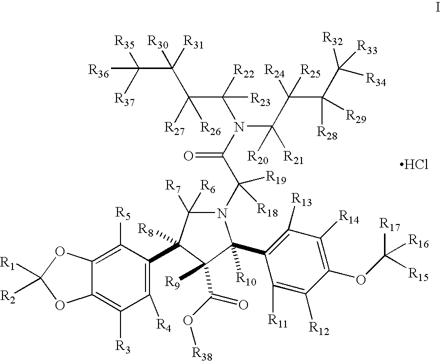
wherein R₁-R₃₈ are independently selected from H and D; and
the abundance of deuterium in R₁-R₃₈ is at least 3%.

2. A deuterium-enriched compound of claim 1, wherein the abundance of deuterium in R₁-R₃₈ is selected from at least 3%, at least 8%, at least 12%, at least 16%, at least 20%, at least 23%, at least 28%, at least 32%, at least 36%, at least 40%, at least 43%, at least 48%, at least 52%, at least 56%, at least 60%, at least 63%, at least 68%, at least 72%, at least 76%, at least 80%, at least 83%, at least 88%, at least 92%, at least 96%, and 100%.

3. A deuterium-enriched compound of claim 1, wherein the abundance of deuterium in R₉, R₁₈, and R₁₉ is selected from at least 33%, at least 67%, and 100%.

4. A deuterium-enriched compound of claim 1, wherein the abundance of deuterium in R₃-R₅, and R₁-R₁₄ is selected from at least 14%, at least 29%, at least 43%, at least 57%, at least 71%, at least 86%, and 100%.

5. A deuterium-enriched compound of claim 1, wherein the abundance of deuterium in R₁-R₂, R₆-R₈, R₁₀, R₁₅-R₁₇, R₂₀, and R₃₇ is selected from at least 9%, 18%, at least 27%, at least 36%, at least 45%, at least 56%, at least 64%, at least 73%, at least 82%, at least 91%, and 100%.

6. A deuterium-enriched compound of claim 1, wherein the abundance of deuterium in R₃-R₅, R₉, R₁₁-R₁₄, R₁₈, R₁₉ is selected from at least 10%, at least 20%, at least 30%, at least 40%, at least 50%, at least 60%, at least 70%, at least 80%, at least 90%, and 100%.

7. A deuterium-enriched compound of claim 1, wherein the abundance of deuterium in R₁-R₂, R₆-R₈, R₉, R₁₀, R₁₅-R₁₇, R₁₈, R₁₉, R₂₀, and R₃₇ is selected from at least 7%, at least 14%, at least 21%, at least 29%, at least 36%, at least 43%, at least 50%, at least 57%, at least 64%, at least 71%, at least 79%, at least 86%, at least 93%, and 100%.

8. A deuterium-enriched compound of claim 1, wherein the abundance of deuterium in R₁-R₂, R₃-R₅, R₆-R₈, R₁₀, R₁₁-R₁₄, R₁₅-R₁₇, R₂₀, and R₃₇ is selected from at least 6% , at least 11%, at least 17%, at least 22%, at least 28%, at least 33%, at least 39%, at least 44%, at least 50%, at least 56%, at least 61%, at least 67%, at least 72%, at least 78%, at least 83%, at least 89%, at least 94%, and 100%.

9. A deuterium-enriched compound of claim 1, wherein the abundance of deuterium in R₁-R₂ is selected from at least 50% and 100%.

10. A deuterium-enriched compound of claim 1, wherein the abundance of deuterium in R₁₅-R₁₇ is selected from at least 33%, at least 67%, and 100%.

11. A deuterium-enriched compound of claim 1, wherein the abundance of deuterium in R₂₀-R₃₇ is selected from at least 6% , at least 11%, at least 17%, at least 22%, at least 28%, at least 33%, at least 39%, at least 44%, at least 50%, at least 56%, at least 61%, at least 67%, at least 72%, at least 78%, at least 83%, at least 89%, at least 94%, and 100%.

12. A deuterium-enriched compound of claim 1, wherein the compound is selected from compounds 1-10 of Table 1.

13. A deuterium-enriched compound of claim 1, wherein the compound is selected from compounds 11-20 of Table 2.

14. An isolated deuterium-enriched compound of formula I or a pharmaceutically acceptable salt thereof: 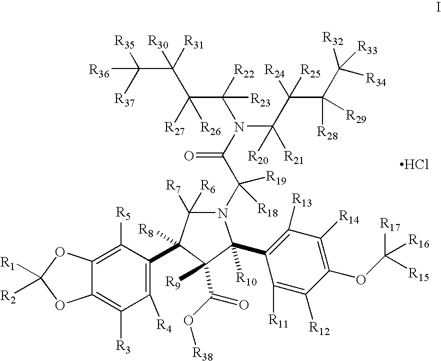
wherein R₁-R₃₈ are independently selected from H and D; and
the abundance of deuterium in R₁-R₃₈ is at least 3%.

15. An isolated deuterium-enriched compound of claim 14, wherein the compound is selected from compounds 1-10 of Table 1.

16. An isolated deuterium-enriched compound of claim 14, wherein the compound is selected from compounds 11-20 of Table 2.

17. A mixture of deuterium-enriched compounds of formula I or a pharmaceutically acceptable salt thereof: 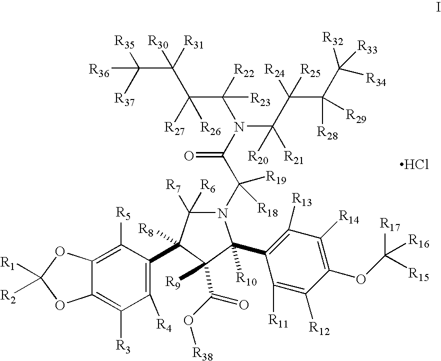
wherein R₁-R₃₈ are independently selected from H and D; and
the abundance of deuterium in R₁-R₃₈ is at least 3%.

18. A mixture of deuterium-enriched compounds of claim 17, wherein the compounds are selected from compounds 1-10 of Table 1.

19. A mixture of deuterium-enriched compounds of claim 17, wherein the compounds are selected from compounds 11-20 of Table 2.

20. A pharmaceutical composition, comprising: a pharmaceutically acceptable carrier and a therapeutically effective amount of a compound of claim 1 or a pharmaceutically acceptable salt form thereof.

21. A method for treating prostate cancer comprising: administering, to a patient in need thereof, a therapeutically effective amount of a compound of claim 1 or a pharmaceutically acceptable salt form thereof.
